(19) United States
(12) Patent Application Publication
(10) Pub. No.: US 2022/0346707 A1
(43) Pub. Date: Nov. 3, 2022

(54) Handheld Closed-Loop Automatic Insulin Delivery System

(71) Applicants: Carlo Giovanni Traverso, Newton, MA (US); Hen Wei Huang, Watertown, MA (US); Siheng You, Somerville, MA (US); Erin Raftery, Boston, MA (US)

(72) Inventors: Carlo Giovanni Traverso, Newton, MA (US); Hen Wei Huang, Watertown, MA (US); Siheng You, Somerville, MA (US); Erin Raftery, Boston, MA (US)

(21) Appl. No.: 17/735,719

(22) Filed: May 3, 2022

Related U.S. Application Data
(60) Provisional application No. 63/183,451, filed on May 3, 2021.

(51) Int. Cl. A61B 5/00; A61M 5/20; A61M 5/315; A61B 5/145; A61B 5/151; A61B 5/157

(52) U.S. Cl. CPC A61B 5/4839; A61M 5/2053; A61M 5/31546; A61B 5/14532; A61B 5/151; A61B 5/157; A61M 2205/3592; A61M 2207/00

(57) ABSTRACT

An all-in-one insulin pen includes a handheld housing that houses a glucose testing module, an insulin delivery module, a wireless communication module, and a microcontroller. The glucose testing module tests a one-time glucose concentration of a patient. The insulin delivery module automatically administers an insulin bolus to the patient based at least in part on the one-time glucose concentration.

### CROSS-REFERENCE TO RELATED PATENT APPLICATION(S)

The present application claims priority to U.S. provisional application No. 63/183,451, filed on May 3, 2021, entitled “A HANDHELD CLOSED-LOOP AUTOMATIC INSULIN DELIVERY SYSTEM,” which is incorporated herein by reference in its entirety.

### BACKGROUND

Diabetes is a chronic disease that affects 34 million people in the US and 422 million people worldwide with rapidly increasing incidence rates. It is associated with significant morbidity and is one of the top ten leading causes of death worldwide. Glycemic control is a primary goal of therapy in diabetes, as it reduces complications, co-morbidities, and mortality, and insulin is one of the therapeutic classes used to achieve this. Insulin is a life-saving therapy in type 1 diabetes and is prescribed as stand-alone or combination therapy for type 2 diabetes, with approximately 25 percent of people with diabetes using insulin. Insulin use for glycemic control involves not only self-injections, often multiple per day, but also routine monitoring of glucose levels, commonly from finger stick capillary blood self-sampling. Moreover, insulin regimens can be extremely complex. A long-acting basal insulin is generally prescribed as a fixed dose taken once or twice daily. A different, short-acting, insulin can be used as a bolus both to mitigate the blood glucose rise after carbohydrate intake, and as a correction for glucose levels above target.

The insulin amount that dampens the glycemic excursion from a carbohydrate load is either a fixed dose or based on an insulin-to-carbohydrate ratio prescription. Basing on an insulin-to-carbohydrate ratio offers more precision, since carbohydrate content per meal usually varies. To safely dose a single insulin injection, a patient typically uses a lancing needle to extract a droplet of blood, prepares a glucometer and places the sample on a test strip for blood glucose measurement, and estimates the amount of carbohydrates they will consume. Then they calculate how much insulin to administer based on their planned carbohydrate load and their present blood glucose level and perform a self-injection. A patient may perform this painful sequence three or more times daily.

### SUMMARY

Glycemic control through titration of insulin dosing remains the mainstay of diabetes mellitus treatment. Insulin therapy is generally divided into dosing with long- and short-acting insulins where long-acting insulins provide basal coverage and short-acting support glycemic excursions associated with eating. The dosing of short-acting insulin often involves several steps for the user including a blood glucose measurement and an estimation of a potential carbohydrate load to determine a safe and appropriate insulin dose.

The inventors have recognized that there is a significant burden on diabetes patients to manage their treatment. Patients typically measure their blood glucose concentrations, determine insulin dosing, and administer an insulin dose using two or three separate devices. The complicated nature of treatment manifests in substantial effects on treatment adherence.

To address this issue, the inventors have developed a comprehensive all-in-one insulin delivery system that streamlines treatment operations that peripheral devices use for safe insulin administration. In turn, this system significantly reduces the complexity and time required for titration of insulin. The system is autonomous and provides ease of use and accurate dosing of insulin.

Embodiments of the invention include an all-in-one insulin pen. The all-in-one insulin pen includes a handheld housing. The all-in-one insulin pen includes a glucose testing module disposed in the handheld housing. The glucose testing module tests a one-time glucose concentration of a patient. The glucose testing module includes a vacuum chamber, a lancet apparatus, and a glucose sensor. The all-in-one insulin pen also includes an insulin delivery module disposed in the handheld housing to automatically administer an insulin bolus to the patient. The insulin delivery system includes an insulin pump and an injection motor. The all-in-one insulin pen also includes a wireless communication module disposed in the handheld housing to communicate with an external device. The external device is capable of (i) setting an insulin dose to be administered by the insulin delivery system and (ii) recording glucose concentration and insulin dose history measured and administered, respectively, by the system. The all-in-one insulin pen also includes a microcontroller disposed in the handheld housing to initiate and control glucose testing, insulin delivery, and wireless communication.

In a version, the handheld housing may have a volume of less than about 300 cubic centimeters. The glucose sensor may be configured to calculate an insulin dose using the one-time glucose concentration of the patient. The wireless communication module may be configured to send the insulin dose calculated to the external device. The external device may be configured to set the insulin dose to be administered using the insulin dose calculated and at least one additional factor input from the patient.

In a version, the all-in-one insulin pen may also include a micropump operably coupled to the vacuum chamber to provide a vacuum to the vacuum chamber when the vacuum chamber forms a seal with a portion of skin of the patient. The vacuum in the vacuum chamber may cause the portion of skin to increase in height by about 2 mm to about 10 mm. The glucose testing module may be configured to hold the vacuum for about 3 seconds to about 5 seconds before lancing the portion of skin and about 3 seconds to about 5 seconds after lancing the portion of skin. The glucose testing module may be further configured to release the vacuum after about 3 seconds to about 5 seconds after lancing, wait 3 seconds to about 5 seconds at atmospheric pressure, and then again create the vacuum in the vacuum chamber. The lancet apparatus may include a spring-loaded actuator. The lancet apparatus may include a motorized lancet. The lancet apparatus may include at least one of a solenoid actuator or a DC-micro motor. The external device may initialize the all-in-one insulin pen.

In a version, the glucose testing module further includes a single-use testing strip, and the insulin delivery module further includes a single-use needle. The glucose testing module may be configured to extract at least 1 microliter of blood from the patient.

Another embodiment of the present technology includes a method of making an all-in-one insulin pen. The all-in-one insulin pen includes a vacuum chamber, a micropump, a lancet apparatus, a glucose sensor, an insulin pump, an injection motor, a wireless communication module, a microcontroller unit, and a handheld housing. The method includes operably coupling the microcontroller unit with the micropump, lancet apparatus, glucose sensor, insulin pump, injection motor, and wireless communication module. The method also includes operably coupling the micropump with the vacuum chamber. The method also includes operably coupling the insulin pump with the injection motor. The method also includes assembling the all-in-one insulin pen into the handheld housing.

In a version of the method, the all-in-one insulin pen includes a handheld housing, a glucose testing module disposed within the handheld housing, and an insulin delivery module disposed within the handheld housing. The method may include placing the all-in-one insulin pen against a portion of skin of a user and actuating the all-in-one insulin pen. While the all-in-one insulin pen is continuously held against the portion of skin, a one-time blood glucose concentration of the user may be tested by the glucose testing module, an insulin dose may be calculated based on the blood glucose concentration, and the insulin dose may be administered to the user by the insulin delivery module. In a version, the glucose testing module may include a vacuum chamber to form a seal against the portion of skin and a micropump to form a vacuum in the vacuum chamber while the one-time blood glucose concentration of the user is tested. The vacuum may cause the portion of skin to increase in height by about 2 mm to about 10 mm.

All combinations of the foregoing concepts and additional concepts discussed in greater detail below (provided such concepts are not mutually inconsistent) are part of the inventive subject matter disclosed herein. In particular, all combinations of claimed subject matter appearing at the end of this disclosure are part of the inventive subject matter disclosed herein. The terminology used herein that also may appear in any disclosure incorporated by reference should be accorded a meaning most consistent with the particular concepts disclosed herein.

### BRIEF DESCRIPTIONS OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally and/or structurally similar elements).

FIG. 1A shows a first view of an all-in-one vacuum/strip pen system.

FIG. 1B shows an exploded view of the all-in-one vacuum/strip pen system in FIG. 1A.

FIG. 2 shows cross-section views of the all-in-one system shown in FIG. 1A during operation.

FIG. 3A is a photograph of an all-in-one system.

FIG. 3B is a photograph of a back view of the all-in-one system in FIG. 3A.

FIG. 3C is a photograph of a front view of the all-in-one system in FIG. 3A.

FIG. 3D is a photograph of a side view of the all-in-one system in FIG. 3A.

FIG. 4A shows the all-in-one system in FIG. 3A without its consumable components or vacuum housing.

FIG. 4B shows the all-in-one system in FIG. 3A with its consumable components but without its vacuum housing.

FIG. 4C shows the all-in-one system in FIG. 3A with its consumable components and vacuum housing.

FIG. 5 shows part of a circuit diagram in the all-in-one system shown in FIG. 3.

FIG. 6 shows a power supply circuit diagram in the all-in-one system shown in FIG. 3.

FIG. 7A is a graph of the vacuum effect on skin stretching.

FIG. 7B shows vacuum pressure versus pulse width modulation (PWM) duty cycle.

FIG. 8 is a graph of the chance of getting blood drops under various conditions.

FIG. 9 is a graph of the chance of getting a blood drop larger than 1 μL under various conditions.

FIG. 10 shows a cyclic voltammetry curve of an onboard integrated chip potentiostat and a commercially-available testing strip.

FIG. 11A shows a characterization curve of the glucose meter with the detection range from 100 to 550 mg/dL.

FIG. 11B shows validation of the developed glucose meter and the comparison with the commercial glucose meter.

FIG. 12 is a graph of the time to pump 3 units of insulin in three different swine thighs at various pumping speeds.

FIG. 13A shows residual drops versus the pumping speed, measured at 5 and 10 seconds after the motor in the all-in-one system stops.

FIG. 13B shows pumping time of the developed insulin pump versus the insulin dose.

### DETAILED DESCRIPTION

A variety of technologies have been developed to help patients living with diabetes manage glycemia, prevent complications, and improve quality of life. Insulin pumps, for example, automate insulin dose calculations and can deliver insulin both continuously and on demand. Continuous glucose monitoring (CGM) technology has drastically reduced the number of finger sticks and revolutionized the amount of blood glucose data that can be collected. More recently, hybrid devices, also known as artificial pancreases, have been approved that both monitor glucose and deliver insulin automatically. However, these devices are expensive, require extensive patient training, are worn continuously to work, and are accessible to a small percentage of patients, usually those with type 1 diabetes. Insulin pens, which are simpler, more accurate and patient-preferred over vial and syringe, are in broad use in both type 1 and type 2 diabetes. New “smart” pens can wirelessly communicate with a CGM device to acquire blood glucose information and automatically calculate required insulin doses for blood glucose correction. However, there remains a need for more complete integration of the processes of measuring blood glucose, and insulin delivery, ideally in a single, easy-to-use device that can be used by the millions of people using insulin.

An all-in-one insulin pen system (also called an all-in-one pen, pen, all-in-one device, or device herein) senses a patient's glucose level, estimates the insulin dose, and delivers the dose of insulin accurately and automatically. The all-in-one pen includes a lancing device, glucose meter, and insulin pump in one device. In an example, this all-in-one system is compatible with commercially available consumable components, including commercial lancets, testing strips, and insulin needles. Any of these consumable components may be single-use components that are changed each time the all-in-one pen is used. The single-use components may be used for one-time measurements of glucose concentration. As an example, the all-in-one pen can use OneTouch testing strips, OneTouch lancets, and MedtFine needles.

With this system, the patient may place the all-in-one pen onto an area of the patient's skin targeted for insulin injection to measure the patient's blood glucose, measure blood glucose, calculate an insulin dose based on the blood glucose measurement, and administer the calculated insulin dose with the pen. The pen may be actuated by mechanical user input, e.g., by pressing a button on the device. Alternatively, or additionally, the pen may be actuated using an external device, such as a smartphone or a laptop, which is wirelessly connected to the pen.

Conventionally, patients measure blood glucose levels by manually pricking their finger using a lancet to extract blood and transferring the blood onto a testing strip. To obtain a sufficient blood volume for an accurate blood glucose measurement, patients usually (1) warm their hands to increase blood flow before lancing and (2) squeeze their fingers to expel blood after lancing.

To automate blood glucose measurements, an embodiment of the all-in-one pen system is equipped with a motor-controlled lancet, an actuator to actuate the motor controlling the lancet, and a vacuum pump to facilitate blood extraction. The vacuum pump and motorized lancet reduce the patient's finger prick pain and discomfort. The actuator may be a solenoid actuator or another electrical or mechanical actuator such as a DC-micro motor. In another embodiment, the lancet includes a spring-loaded actuator. In another embodiment, the all-in-one pen may not include a vacuum pump, and instead may form a vacuum in the vacuum chamber via other means, such as with a suction cup.

To further automate the procedure, the all-in-one pen system may be equipped with a motor to insert the insulin needle into skin and/or an insulin pump to deliver insulin subcutaneously. As an example, the all-in-one system may include three motors: one to drive the lancet, one to drive the insulin needle injection, and one to drive insulin delivery via the pump.

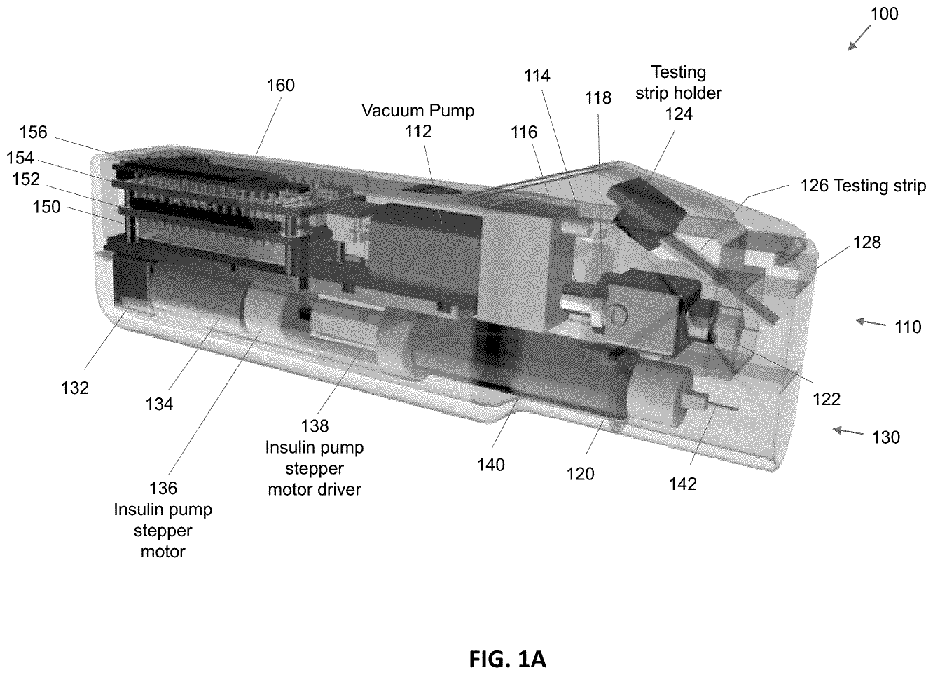

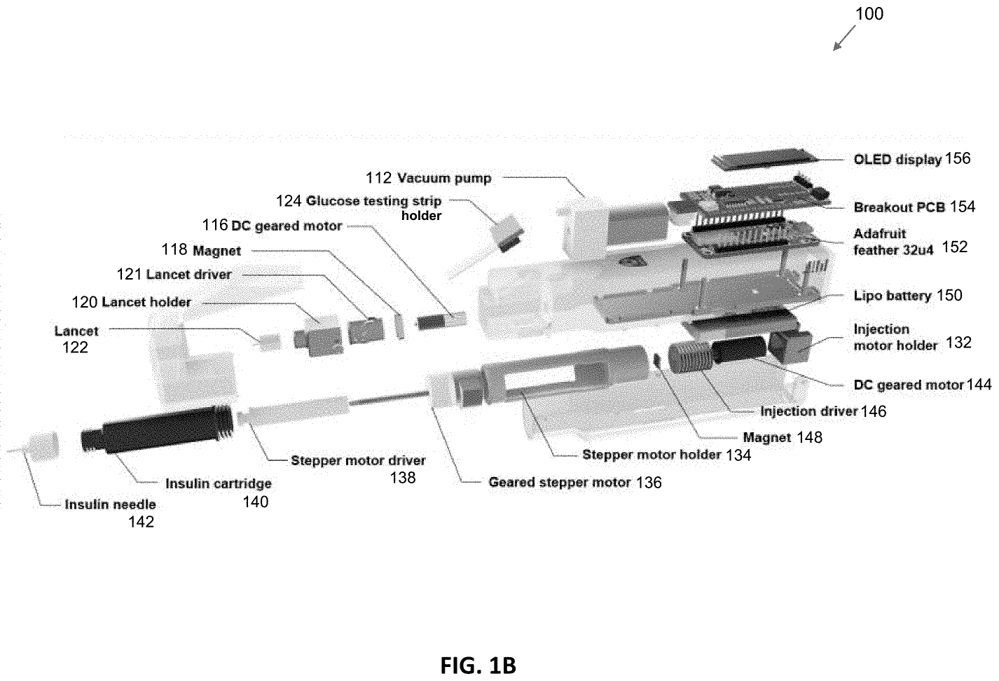

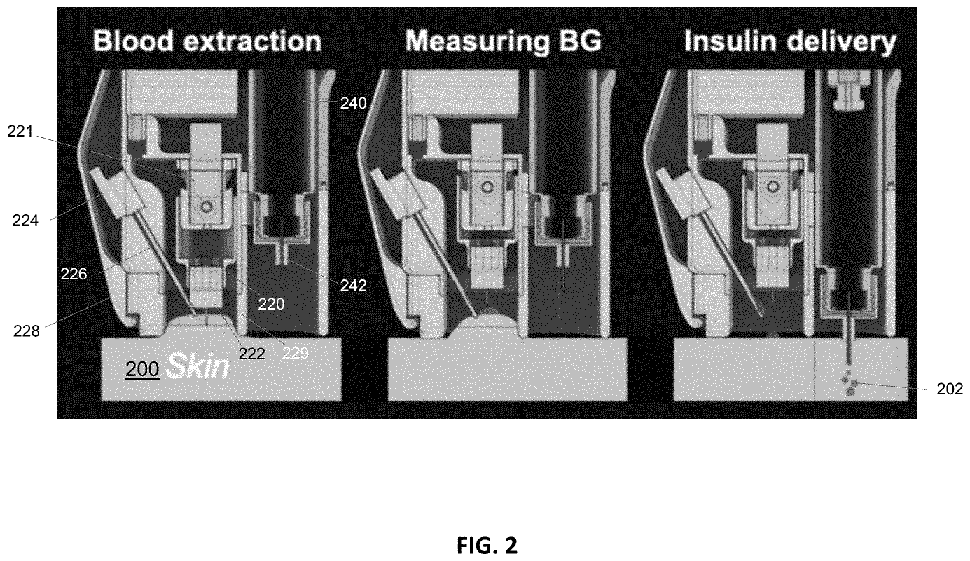

FIGS. 1A and 1B show a perspective view and an exploded view, respectively, of the all-in-one pen 100. The all-in-one pen facilitates the processes of blood glucose measurement, insulin dose calculations, and insulin self-injections usually used for routine diabetes management. The handheld all-in-one pen seamlessly integrates a vacuum-assisted lancing device, a glucometer with test strip, and an insulin delivery pump into a single system. The all-in-one pen system automates procedures of blood sample collection, glucose measurement, and insulin delivery. This device is fully compatible with commercially available lancets, testing strips, and insulin needles, in order to accelerate clinical translation. The device's automated approach can significantly reduce patient time burden, and consequently has the potential to improve medication adherence and diabetes management.

FIG. 1A depicts the outer housing 160 as being translucent, but the outer housing 160 may alternatively be opaque or semi-translucent. The all-in-one pen includes a testing module 110 (also called a glucose testing module) and an insulin delivery module 130 (also called an insulin delivery system). The outer housing 160 may be dimensioned so that it can be held in one hand. As an example, the outer housing 160 may have a volume of less than about 300 cubic centimeters. When the all-in-one pen 100 is placed against a patient's skin, the vacuum pump 112 creates a vacuum in the chamber 128 that is sealed against the patient's skin via vacuum port 114. The vacuum induces skin stretching, a local increase of blood flow, and an analgesic effect.

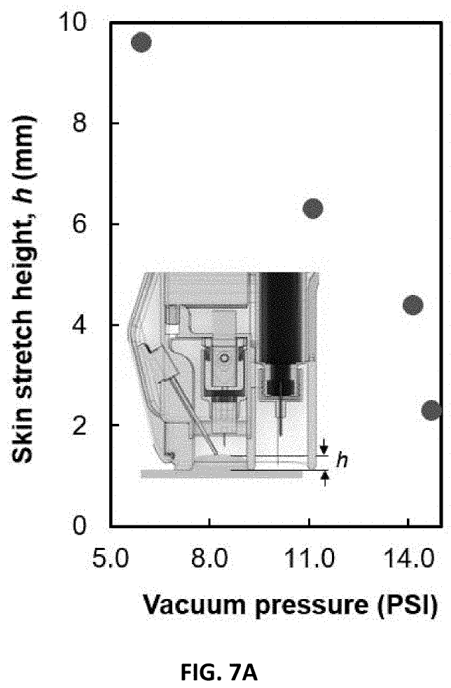

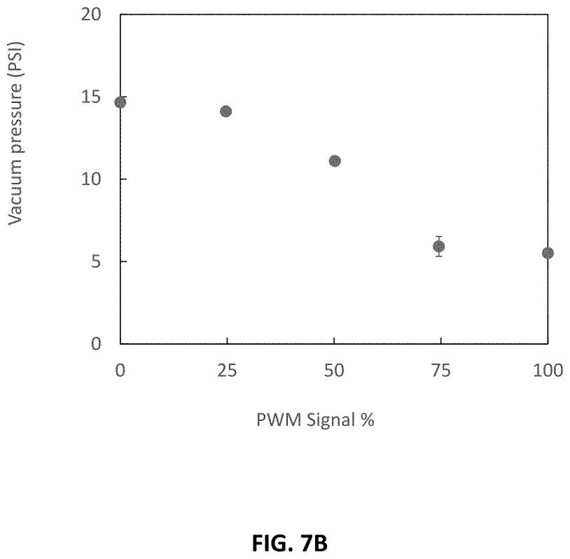

The lancet device (also called a lancet apparatus), including the lancet 122, the lancet holder 120, the lancet driver 121, the magnet 118, and the lancet motor 116, is at least partially disposed inside the vacuum chamber 128. The testing strip holder 124 and the glucose testing strip 126 are likewise at least partially disposed inside the vacuum chamber. The lancet 122 and the glucose testing strip 126 may be wholly disposed in the vacuum chamber 128. Testing strip 126 is removably coupled to the testing strip holder 124. The distance between the skin and the testing strip 126 is controlled by the vacuum induced skin stretching. Lower pressure results in higher skin stretching height as shown in FIG. 7A. Since the skin stretching height can vary from person to person, users may adjust the vacuum pressure to adjust the skin stretching height so that the skin contacts the testing strip 126. Different types of testing strip holders 124 can be interchanged for accommodating different types of testing strips 126.

The lancing device may be activated before, after, or while the vacuum is being formed. Preferably, the lancing device is activated about 3 to about 5 seconds after the vacuum is formed so that the vacuum has time to induce increased blood flow and provide an analgesic effect. The lancet motor 116 provides the system with a high lancing speed and strong enough skin penetration force. The lancet motor 116 may have a speed of at least 500 RPM and a torque of at least 200 g-cm. For example, the lancet motor 116 may be a DC geared motor, a servo motor, a stepper motor, or a solenoid. As an example, the DC geared motor 116 may have a diameter of about 6 mm and length of about 19 mm, an operating voltage of 5 V, an operating current of 30 mA, a motor speed of 500 RPM, and a stall torque of 500 g-cm. The lancing driver 121 converts the unidirectional rotation from the motor 116 to a bi-directional linear movement. As an example, the lancing driver 121 may be 3D printed or manufactured by injection molding. The ring-shaped magnet 118, magnetized across its diameter, is attached to the lancet driver 121 and disposed between the lancet driver 121 and the motor 116 to provide positioning feedback on the position of the lancet. A hall-effect sensor is placed next to the magnet 118 to measure changes in the magnetic field to provide closed-loop control of the lancet actuation. The lancet holder 120 is attached to the lancet driver 121, and a lancet 122 is removably coupled to the lancet holder 120. Alternatively, or in addition to the lancet's lancet motor 116, the lancing device may include a spring-loaded actuator. Alternatively, or in addition to the lancet's lancet motor 116, the lancing device may include a solenoid actuator. Electrical conductors (e.g., electrical wires or ribbon cables) electrically couple the lancet motor 116 to a microcontroller coupled to and/or disposed on a main printed circuit board (PCB) 152.

The vacuum pump 112 (also called a micropump) is electrically coupled to the PCB 152 and/or the same or a different microcontroller. A voltage regulator controls the voltage to the vacuum pump 112 to control the vacuum pressure. In an example, the vacuum pump 112 provides variable vacuum pressure between 15 pounds per square inch (psi) and 5 psi in the vacuum chamber 128. In the example, the variable vacuum pressure may be controlled using an N-type MOSFET mounted on the PCB 152 in series with the vacuum pump 112 and pulse-width modulated (PWM) via the microcontroller to vary the gate to source voltage of the MOSFET from 0 V to 5 V, which modulates the voltage to the vacuum pump 112, and thereby varies the steady-state pressure in the vacuum chamber 128. A pressure sensor (e.g., Honeywell MPRLS) may be disposed inside of the vacuum chamber 128 to measure the pressure and provide feedback for PWM control. Creating a vacuum in the vacuum chamber sealed against a portion of the patient's skin may increase the patient's skin height in the vacuum chamber by up to about 2 mm to 10 mm (in some cases, preferably to about 10 mm) relative to the patient's skin outside the vacuum chamber. The vacuum pump 112 may hold the vacuum for about 3 seconds to about 5 seconds before lancing the patient's skin to increase blood flow at the lancing site. The vacuum pump 112 may also hold the vacuum for about 3 seconds to about 5 seconds after lancing to bring blood to the surface of the skin. In some examples, the microcontroller brings the pressure in the vacuum chamber back to atmospheric pressure (15 psi) after lancing and then create the vacuum in the vacuum chamber again for about 3 seconds to about 5 seconds to increase the size of the blood drop (e.g., about 1 μL or more) for glucose measurement.

Electrical conductors electrically couple the testing strip holder to a blood glucose meter on the PCB 152 (also called a glucose sensor). The blood glucose meter is an integrated chip potentiostat (e.g., system-on-chip TI LMP91000) mounted on the PCB 152. The blood glucose meter can measure the blood glucose level using a commercial testing strip. The blood glucose meter may use cyclic voltammetry (CV) to determine a glucose oxidation peak and then amperometric detection to measure the concentration of glucose (also called a glucose level) at the oxidation peak voltage. Alternatively, the blood glucose meter may skip the CV step and simply use amperometric detection to measure the glucose level. The output current may be amplified by a transimpedance and/or a non-inverting operational amplifier, and the amplified signal may be converted to a digital signal using an analog-to-digital converter (ADC).

The all-in-one pen 100 uses the blood glucose measurement in at least one of three ways. First, the blood glucose measurement may be displayed on the display 156 so that the patient or a healthcare practitioner can read the measurement and determine an appropriate insulin dose accordingly. The display 156 may be an OLED, LED, or LCD screen. Second, a microcontroller on the PCB 152 in the all-in-one pen may use the blood glucose measurement to calculate an appropriate insulin dose to be delivery to the patient. The microcontroller may compute the insulin dose after obtaining the glucose measurement from the potentiostat. The microcontroller may use an equation for calculating an insulin dose using the glucose measurement. For example, the equation coded into the microcontroller may be one or more of those described in Calculating Insulin Dose, Diabetes Teaching Center at the University of California San Francisco, https://dtc.ucsf.edu/types-of-diabetes/type1/treatment-of-type-1-diabetes/medications-and-therapies/type-1-insulin-therapy/calculating-insulin-dose/#formulas (last visited May 3, 2022), which is incorporated by reference herein. Third, a transceiver and antenna on the PCB 152 (also called a wireless communication system or module) may wirelessly send the blood glucose measurement to an external device (e.g., a smart phone, a tablet, or another type of computer). The external device may then display the blood glucose measurement so that the patient or a healthcare practitioner can read the measurement and determine an appropriate insulin dose accordingly. Alternatively, or additionally, the external device may calculate an appropriate insulin dose to be delivery to the patient and wirelessly transmit that information to the all-in-one pen. The external device may also record and store a log or history of blood glucose measurements. The external device may be in the possession of the patient or their healthcare practitioner. The insulin dose calculation encoded in the all-in-one pen system is responsible for high blood sugar correction based on the blood glucose measurement while the calculation encoded in the external device may consider additional factors from the patient or healthcare practitioner, including the patient's carbohydrate intake. The user may manually input an insulin sensitivity factor and an insulin to carbohydrate ratio in the all-in-one pen system and/or the external device as an initialization setting.

The insulin dose is calculated by the microcontroller in the all-in-one pen 100, manually by the patient, or using an external device via the wireless communication system communicating with the external device. The insulin delivery module 130 then delivers the calculated insulin dose to the patient. The insulin module 130 includes an insulin needle 142, a syringe-like insulin cartridge 140, a stepper motor driver 138 with a screw plunger (also called an insulin pump), a stepper motor 136, and a stepper motor holder 134. As an example, the stepper motor 136 may be a two-phase four-wire geared stepper motor with a lead screw. The insulin module 130 also includes a micro-DC geared motor 144 (also called an injection motor) (e.g., having parameters 3 V, 30 mA, 100 RPM), an injection driver 146, a magnet 148 and an injection motor holder 132. Electrical conductors (e.g., electrical wires or ribbon cables) electrically couple the stepper motor 136 to the same or a different microcontroller coupled to and/or disposed on a main printed circuit board (PCB) 152.

The DC geared motor 144 drives the movement of the needle 142 into the patient's subcutaneous tissue with the injection driver 146. The magnet 148 (e.g., a square magnet magnetized through its width) is embedded in the injection motor driver 146 to provide positioning feedback. A hall sensor is disposed next to the magnet 148 (e.g., in the DC motor) to measure changes in the magnetic field induced by the injection motor to provide closed-loop control of the automatic insulin needle injection. Electrical conductors (e.g., electrical wires or ribbon cables) electrically couple the DC geared motor 144 to the same or a different microcontroller coupled to and/or disposed on a main printed circuit board (PCB) 152.

Once the needle is in the tissue, the stepper motor 136 drives the stepper motor driver 138 to administer insulin from the cartridge 140 (also called a reservoir) to the patient via the insulin needle 142. The insulin cartridge 140, stepper motor driver 138, and stepper motor holder 134 may be 3D printed and/or made using injection molding and may be made of durable materials. Pumping rate is controlled by changing the speed of the stepper motor 136. The insulin cartridge 140 may be pre-filled with insulin (e.g., from a commercially available insulin vial using a syringe). The cartridge 140 may be loaded in the all-in-one pen before use. The injection motor driver (e.g., an H-bridge circuit) controlled by the microcontroller may be used to vary the velocity and direction of the DC geared motor 144. The amount of insulin delivered may be displayed on the pen's display 156 for ease of use. The amount of insulin delivered may be transmitted to the external device via the wireless communication system so that this information may be stored as art of the insulin dose history.

All of the electronic components, including those in the testing module 110, the insulin delivery module 130, the wireless communication system, and the microcontroller are powered by one or more rechargeable batteries 150. The rechargeable battery 150 (e.g., lithium-ion or lithium-ion polymer) may be able to supply a current of 500 mA continuously for a fixed period of time. As an example, the rechargeable battery 150 may be a lithium-ion polymer (LIPO) battery. As an example, the rechargeable battery 150 may be recharged by the patient or a healthcare professional when desired by electrically coupling the all-in-one pen to an external power source directly or using inductive coupling.

The all-in-one pen 100 may be assembled by operably coupling the microcontroller unit disposed on the PCB 152 with the pump 112, lancet motor 116, integrated chip glucose sensor on the PCB 152, insulin pump stepper motor 136, injection motor 144, and wireless communication system. The pump 112 may be fluidically coupled with the vacuum chamber via the port 114. The insulin pump and the injection motor may be operably coupled via the PCB 152. All of these components may be assembled into a handheld housing 160.

FIG. 2 shows a process to use the all-in-one pen, with three steps in succession from left panel to right panel. Before using the all-in-one pen, the patient manually loads a testing strip 226 into the test strip holder 224, lancet 222 into the lancet holder 220, and insulin needle 242 onto the insulin cartridge 240. In some versions, part of the housing 228 may be removed so that the patient may more easily load these consumable components into the all-in-one pen. In one example, the all-in-one pen may be initiated by an external device (e.g., a mobile application on a smart phone), with the all-in-one pen receiving a wireless or wired signal from the external device. In some versions, once the patient initiates the procedure, the patient has a set period of time (e.g., 3 seconds to 10 seconds, preferably about 5 seconds) to place the all-in-one pen on their skin 200 at the desired measurement and injection site on the body.

The vacuum pump in the all-in-one system creates a vacuum in the vacuum chamber 229 to induce a local increase of blood flow and an analgesic effect at the site. Then, the motorized lancing driver 221 is actuated so that the lancet 222 pricks the skin at the site. After lancing, the vacuum is maintained in the vacuum chamber 229 sealed against the skin 200 at the site for another period of time (e.g., about three seconds to about five seconds), and then the vacuum chamber 229 is allowed to return to atmospheric pressure. The generated pressure gradient between the stretched skin and the chamber drives the blood sample extraction. After another period of time (e.g., about three to about five seconds) at atmospheric pressure, a second vacuum with the same or higher amplitude is created in the vacuum chamber 229 with the vacuum pump to again stretch the skin at the site to bring more blood to the surface of the skin and to feed the blood drop into the testing strip 226.

The onboard glucose meter measures the blood glucose level. At least one of the all-in-one pen, the external device, or the patient calculates an insulin dose based at least in part on the measured blood glucose level. The injection motors are subsequently actuated to move the needle 242 into the skin 200 and to administer the calculated insulin dose 202 subcutaneously from the insulin cartridge 240. In some versions, the delivered insulin dose and the measured blood glucose are automatically recorded on the external device or on the all-in-one pen.

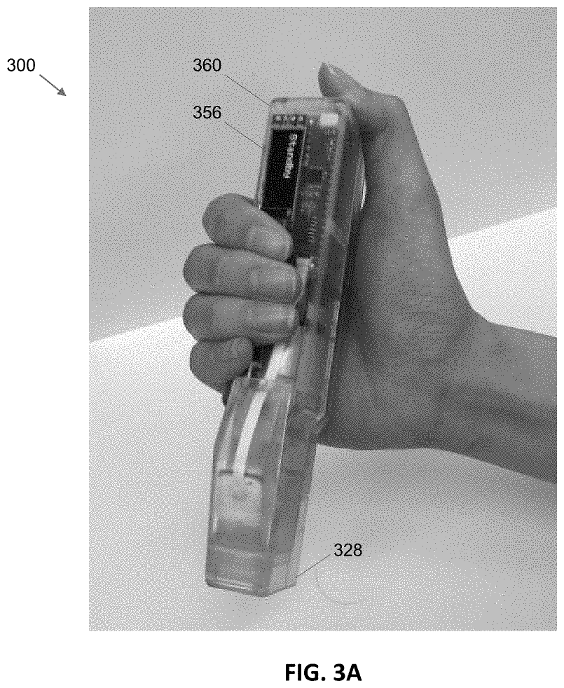

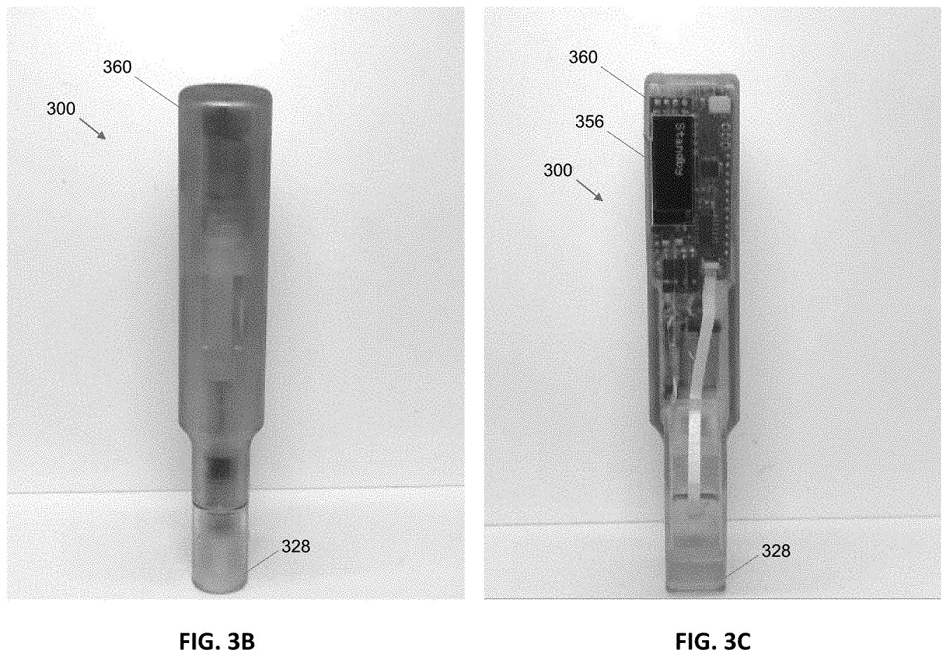

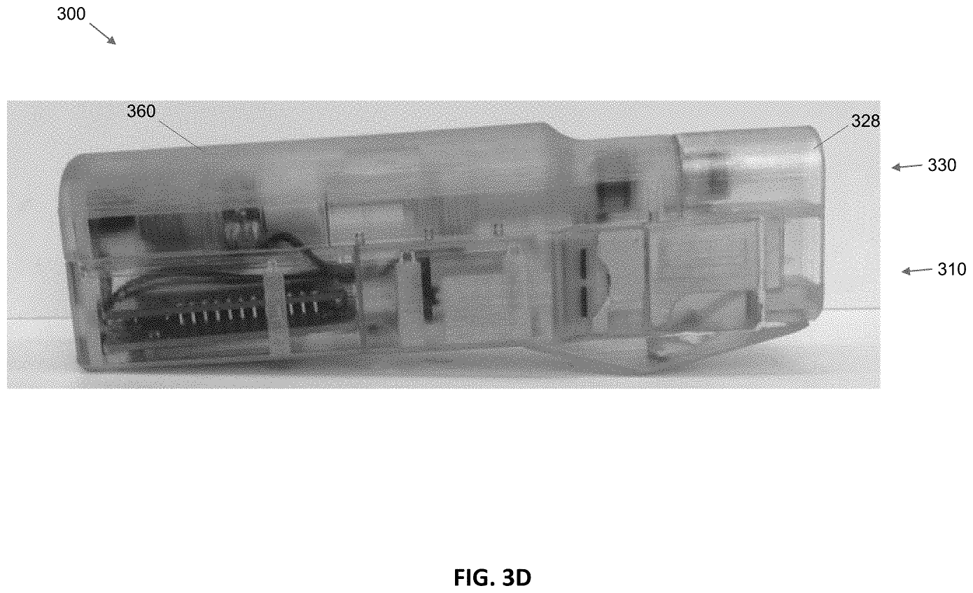

FIGS. 3A-3D show various views of an all-in-one pen 300. The pen 300 is small enough to be held and operated with one hand. The pen 300 includes an outer housing 360, a vacuum chamber 328 that creates a vacuum with a vacuum pump when sealed against a user's skin, and a display 356 electrically coupled to one or more PCBs in the pen 300 to display operating parameters, blood glucose measurements, calculated insulin doses, and current operating mode (e.g., “measuring,” “injecting,” or “standby”). The pen 300 includes a testing module 310 and an insulin delivery module 330.

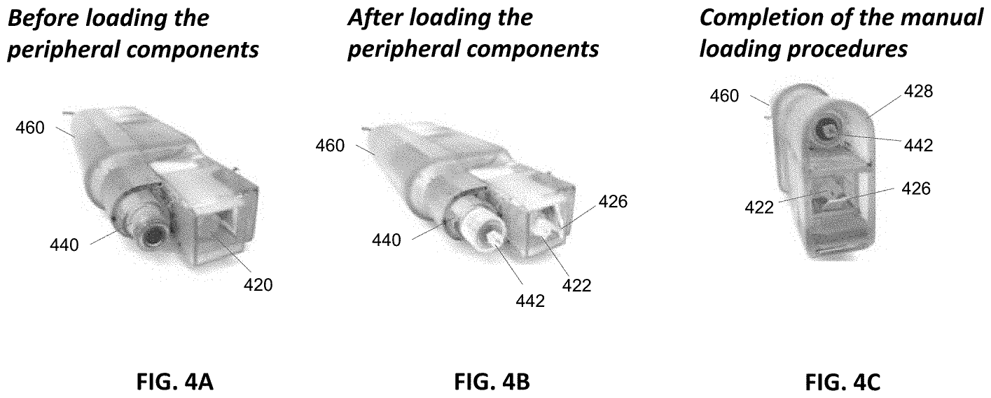

FIGS. 4A-4C show a three-step procedure for preparing an all-in-one pen for operation. First, as shown in FIG. 4A, the outer housing that forms the vacuum chamber 428 is removed so that the lancet holder 420, the test strip holder, and insulin cartridge 440 are easily accessible. Then, as shown in FIG. 4B, the lancet 422 is loaded onto the lancet holder 420, the test strip 426 is loaded onto the test strip holder, and the insulin needle 442 is loaded onto the insulin cartridge 440. Then, as shown in FIG. 4C, the outer housing that forms the vacuum chamber 428 is coupled to the rest of the outer housing 460.

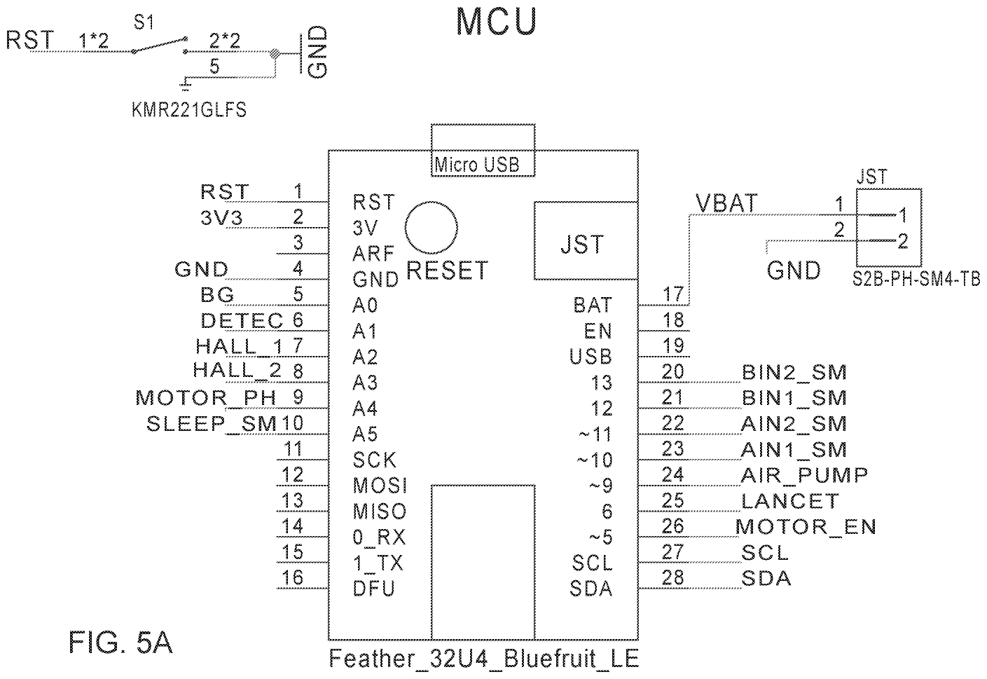

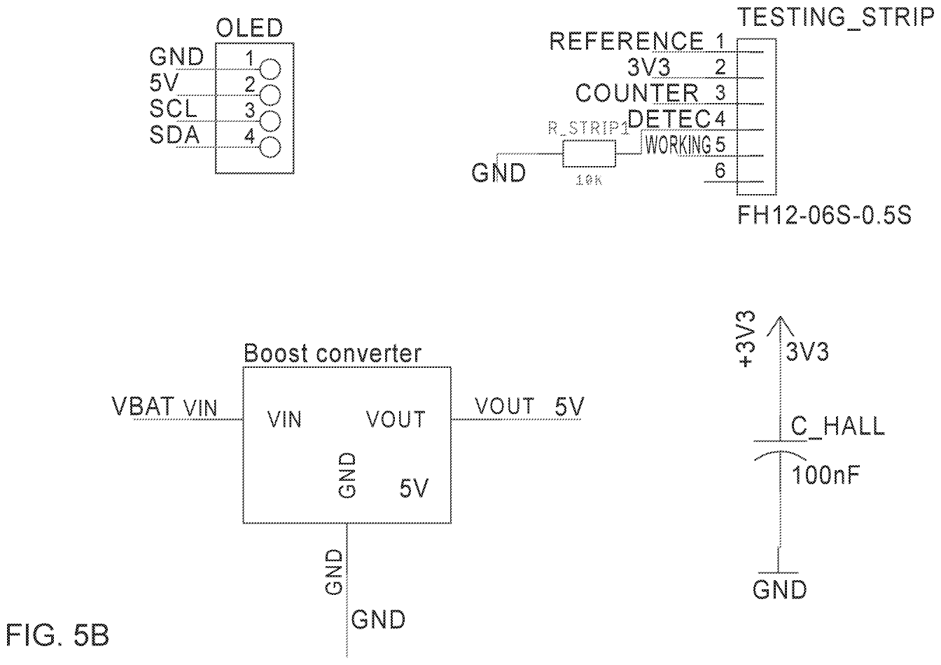

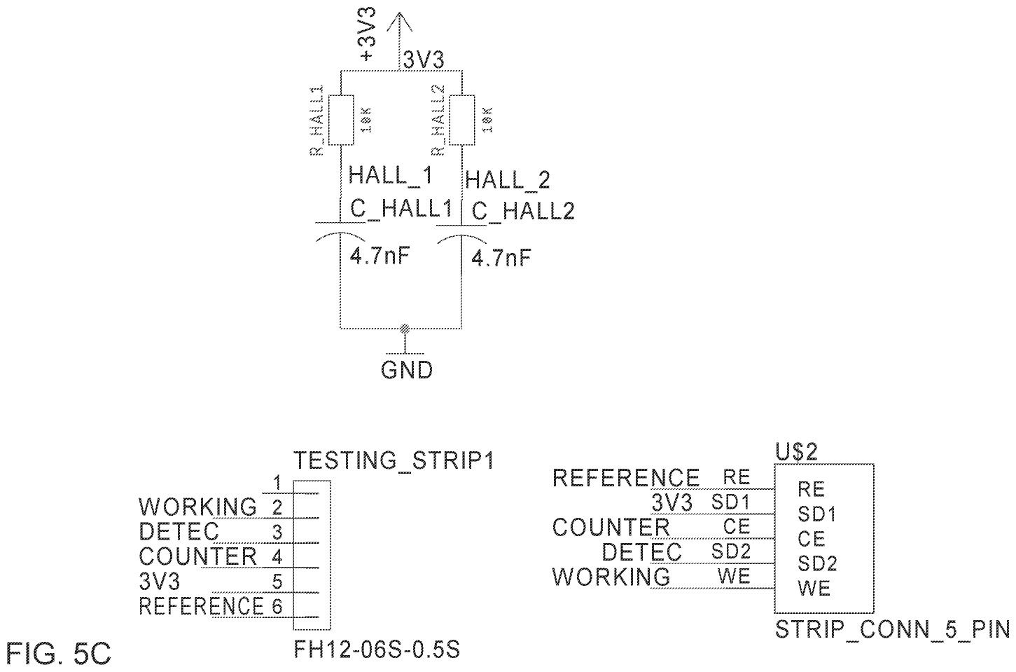

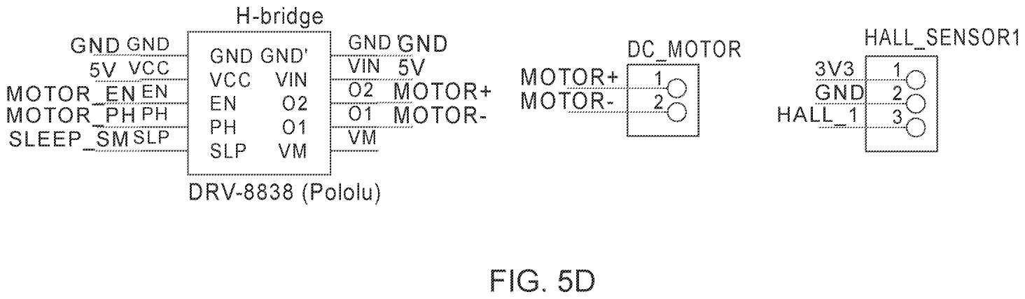

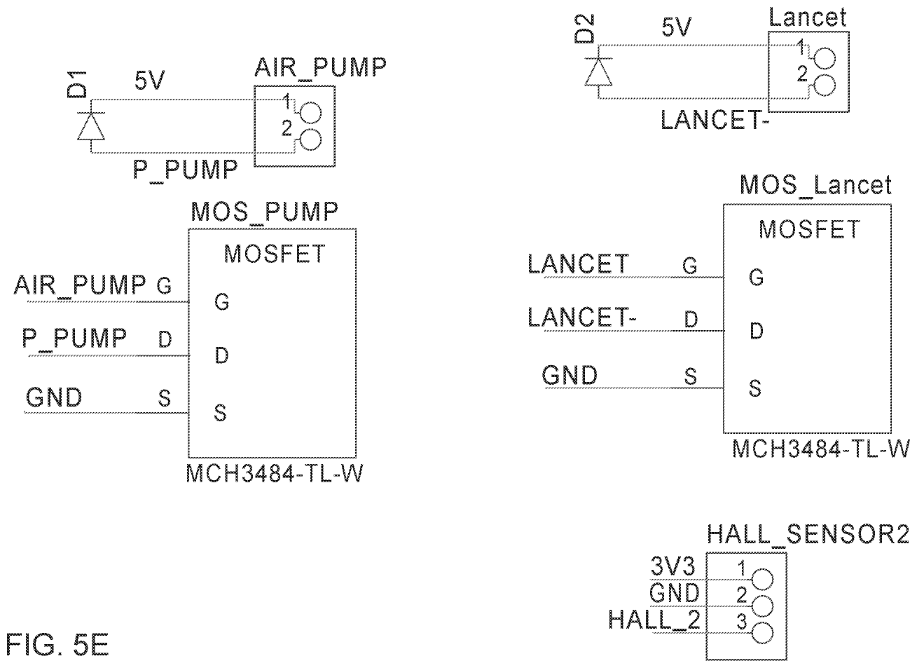

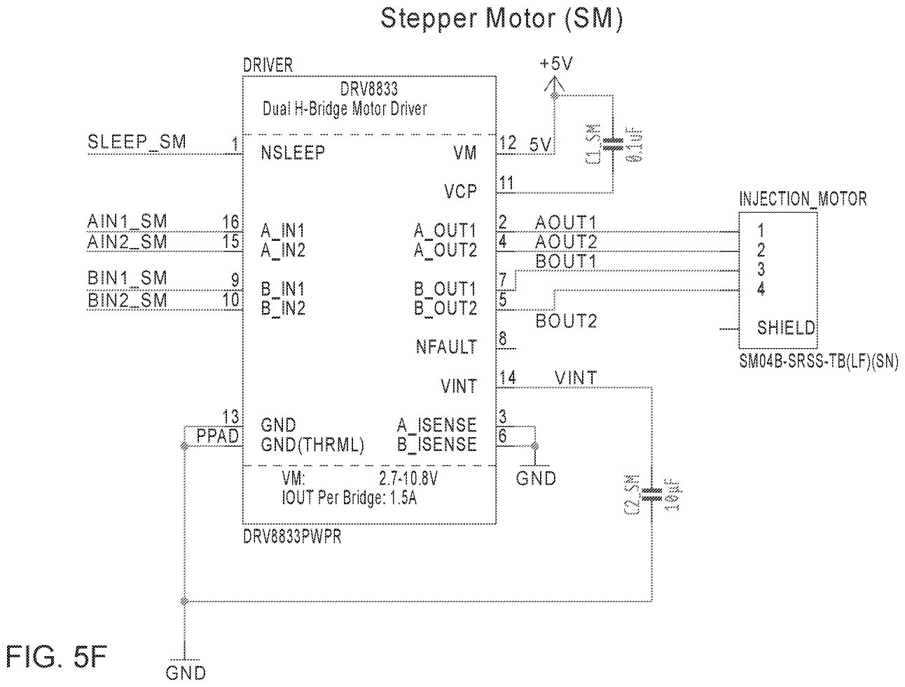

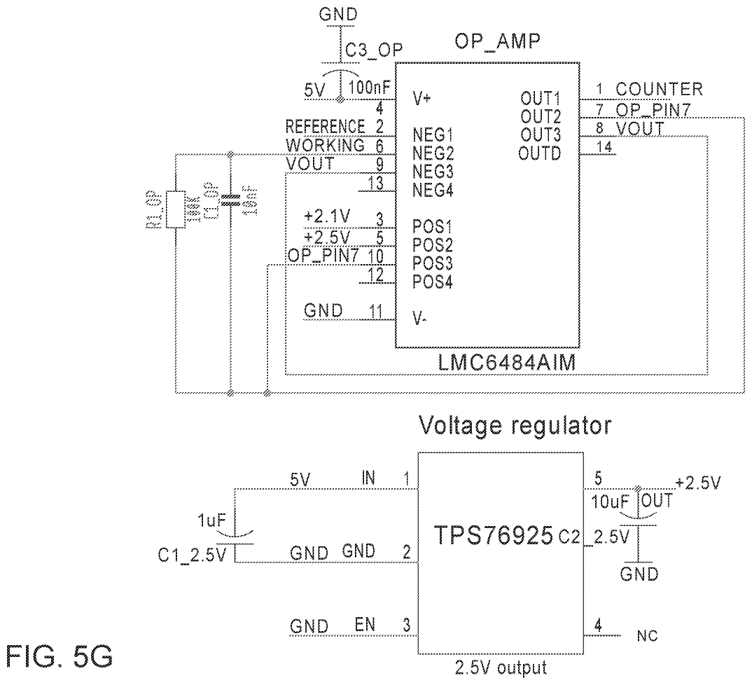

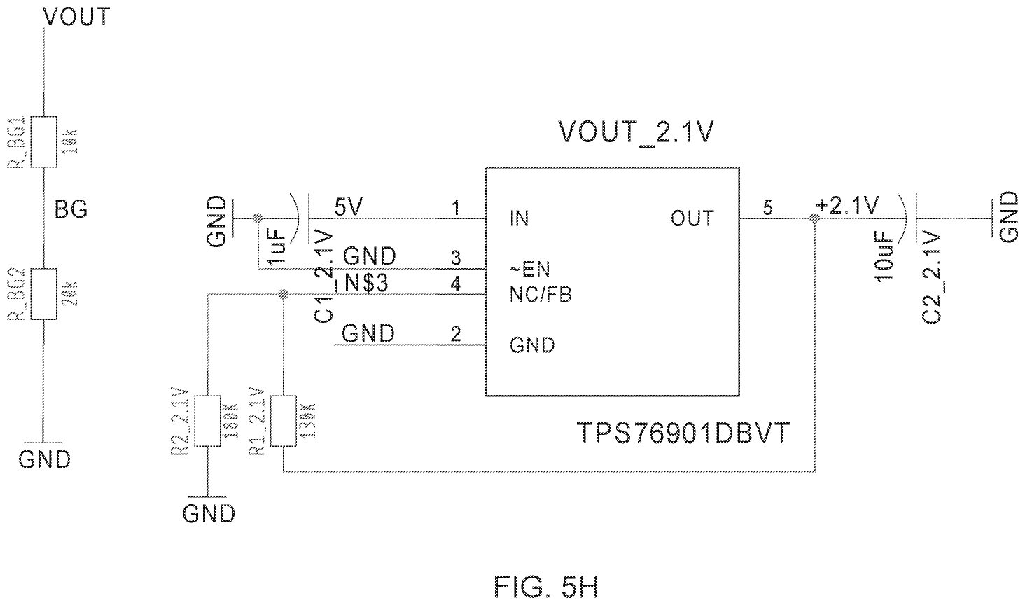

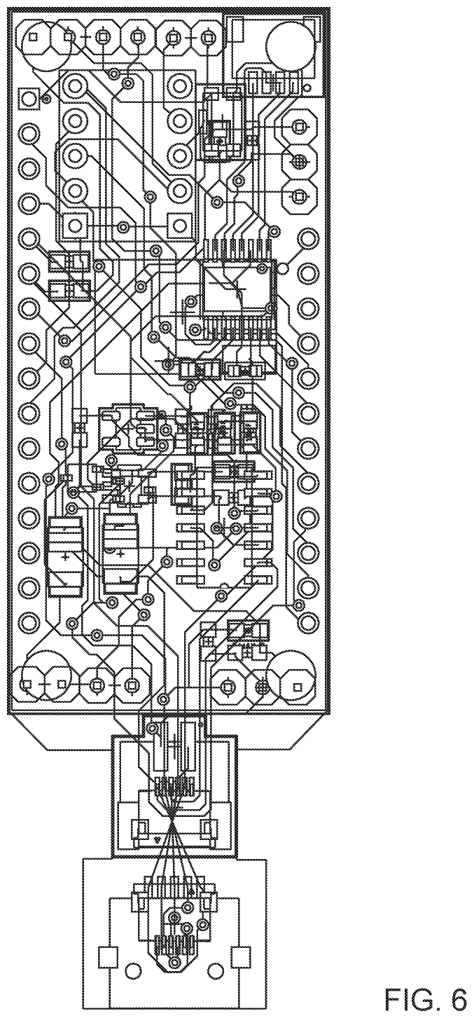

FIGS. 5 and 6 show circuitry that may be present in the all-in-one pen. The circuitry may include the microcontroller, the glucose meter potentiostat, the injection motor driver, the lancing device, the insulin pump, and the OLED screen. The control circuitry may facilitate the automated procedures of obtaining a blood sample, measuring glucose levels, and delivering the calculated insulin bolus via a single pen. The all-in-one pen seamlessly integrates a motorized lancing device, glucose meter, vacuum chamber, injection motor, and insulin pump with control circuitry. The all-in-one pen may also include communication circuitry to communicate with an external device using a wired or wireless connection. For example, the all-in-one pen may include a Bluetooth chip to communicate with a smart phone, as described above.

As an example, an Adafruit feather 32u4 PCB includes a microcontroller and a Bluetooth communication module, and the Adafruit PCB is mounted on the main PCB. The main PCB also includes the potentiostat, multiple MOSFETs for controlling the lancing device and vacuum pump, the stepper motor driver, and the OLED screen.

### Example Vacuum Effect on Blood Extraction

To facilitate automated blood extraction for blood glucose testing, the inventors introduced a vacuum system to induce skin stretching and blood draw before and after lancing, respectively. This sequence is used instead of the conventional testing actions of warming up the hands to increase blood flow and squeezing the finger to accumulate and extract blood in a local region. The stretched skin height is proportional to both the cross-sectional area of the open channel and the vacuum pressure. The inventors controlled the vacuum pressure to modulate the skin stretching height with a fixed cross-sectional area of 2 cm².

Female Yorkshire swine in the range of 60-80 kg were used for the testing blood extraction and blood glucose measurements using the all-in-one vacuum/strip pen systems. The animals were kept on a liquid diet for 24 hours before the procedure and fasted overnight. Pigs were sedated. For blood sampling, an indwelling catheter was placed in the femoral vein under aseptic conditions. Blood glucose level measurements were compared to commercially-available TRUEtrack Blood Glucose Test Strips using a TRUEtrack Meter.

FIGS. 7A and 7B show different measurements related to vacuum pressure in the vacuum chamber used to extract blood. FIG. 7A shows the relationship between the height of the stretch skin in the vacuum chamber and the vacuum pressure. Vacuum pressure was varied between 15 psi and 5 psi in the vacuum chamber, resulting in skin stretching increasing with decreasing pressure up to a skin stretch height of almost 10 μm at 5.5 psi. The results were obtained from porcine ears since the porcine ear has similar mechanical properties to human skin. The stretching height was considered in determining the position of the lancet device, since different skin stretching heights result in different lancing depths. The lancet device was positioned based on the vacuum pressure of 5.5 psi to keep a constant lancing depth and a high skin stretch height.

FIG. 7B shows the vacuum pressure in the chamber sealed against the patient's skin versus the pulse width modulation (PWM) duty cycle used to control the vacuum pressure. The vacuum pressure may be controlled using a PWM duty cycle to vary the voltage to the vacuum pump. In an example, a pressure sensor inside the chamber may measure the pressure in the chamber and the pressure measurement may be used to vary the PWM duty cycle. FIG. 7B shows that as the PWM signal percentage trends toward 100%, the steady-state pressure in the chamber reduces from about 15 psi to about 5 psi.

Blood extraction tests using a commercial Genteel vacuum-assisted lancing device and the all-in-one vacuum/strip system were performed on porcine ears. Four porcine ears were uniformly lanced 20 times in each condition, as a result, four different pigs were lanced 80 times in total. The success of blood extraction and the volumes of extracted blood were recorded subsequently. The lancets of both the Genteel device and the all-in-one system were replaced every three times to maintain sharpness.

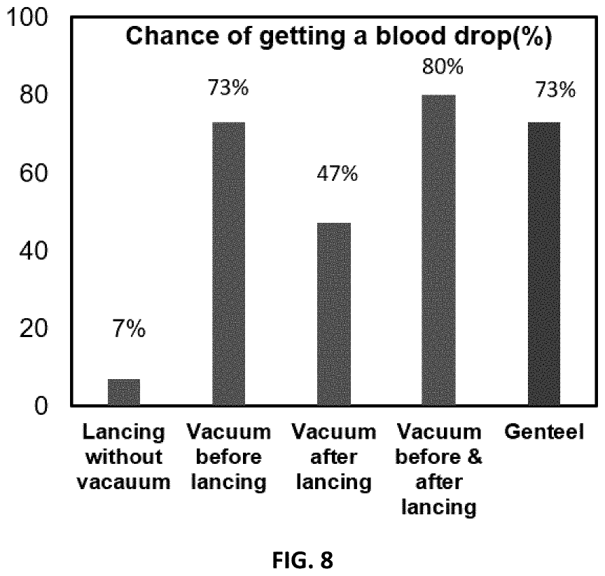

FIG. 8 shows various conditions tested to determine which condition provides a higher chance of forming a blood drop on the patient's skin surface for glucose testing. To verify the vacuum effect on blood extraction, blood extraction was compared under four different conditions: (I) lancing without vacuum, (II) vacuum only before lancing, (III) vacuum only after lancing, and (IV) vacuum before and after lancing. FIG. 8 shows the probability of any blood extraction in the four conditions. As a reference to the success rate of blood extraction from the porcine ears, the results from a Genteel device, an FDA approved painless lancing device that also utilizes vacuum to assist blood extraction, were compared. The results showed that applying a vacuum can significantly increase the chance of successful blood extraction. Moreover, applying a vacuum before lancing resulted in a much higher chance (73%) of a successful blood draw compared to vacuum application after lancing (47%). This observation suggests that the skin stretching via vacuum before lancing is particularly useful as it approximates the action of local heating to induce increased blood flow.

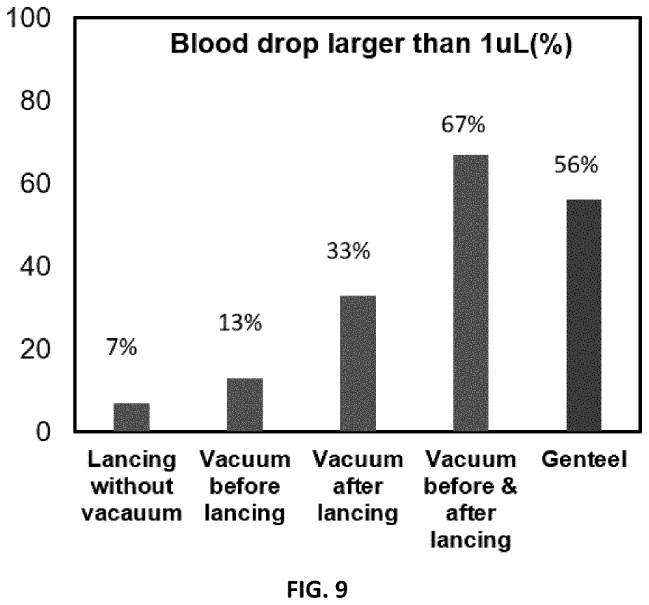

FIG. 9 shows the chances of getting an adequate volume of blood for a blood glucose measurement (e.g., greater than 1 μL) using the same conditions tested in FIG. 8. The results showed that applying vacuum only before lancing does not result in sufficient blood for glucose measurement. To obtain an accurate glucose reading, commercial test strips conventionally use more than 1 μL of blood. Applying vacuum before and after lancing provides a significantly higher chance of getting a blood drop with a volume larger than 1 μL as compared to only applying vacuum before lancing. As a reference to the success rate of blood extraction from the porcine ears, the results from a Genteel device, an FDA approved painless lancing device that also utilizes vacuum to assist blood extraction, were compared. It is worth noting that a hot pack and hand-squeezing were used together to warm up the porcine ears before lancing with the Genteel device. Without these procedures, it was impossible to extract blood from the porcine ears with the Genteel device. In comparison, the all-in-one device shows similar blood draw performance to the commercial Genteel device without any extra warming procedures.

### Example Blood Glucose Meter

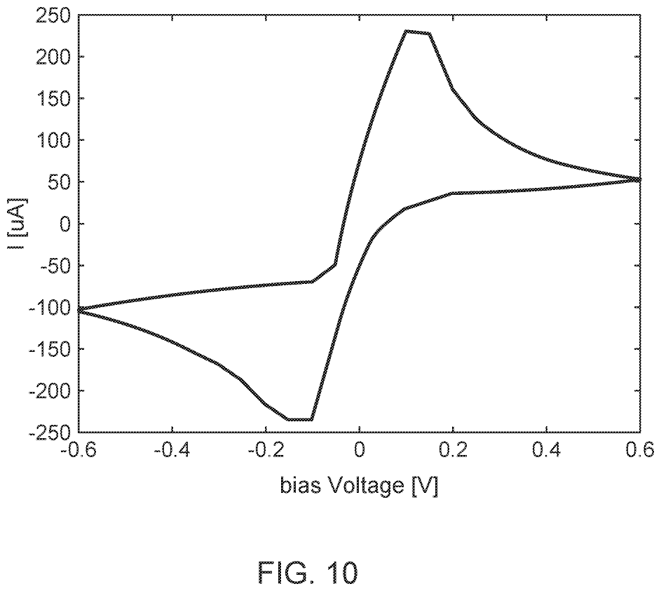

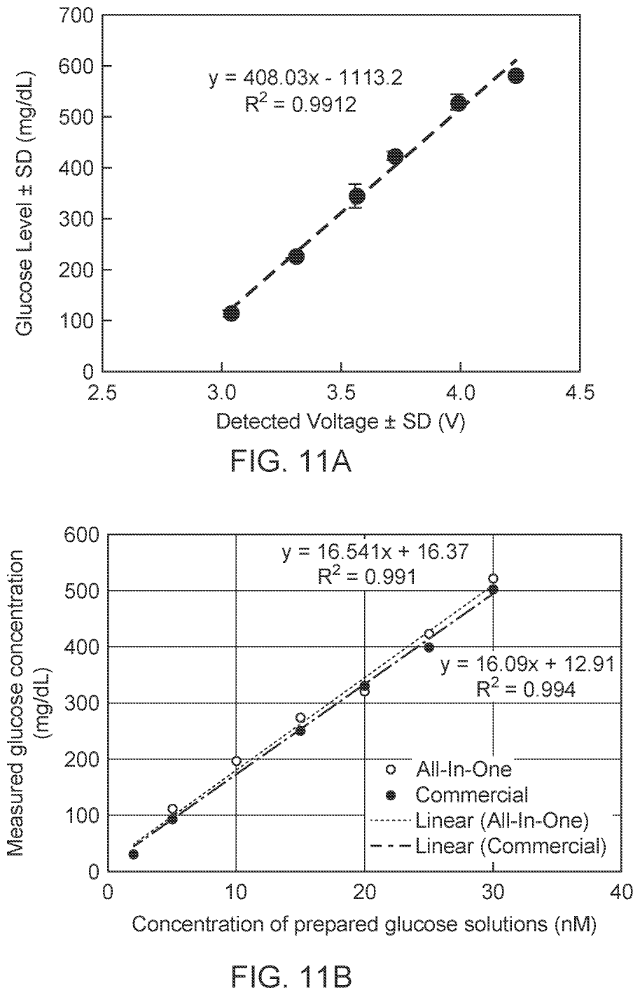

Characterization of the onboard glucose meter in an example all-in-one pen is shown in FIGS. 10, 11A, and 11B and described below.

FIG. 10 shows a cyclic voltammetry curve measured by an integrated chip potentiostat on the all-in-one pen and a commercially-available testing strip. A blood glucose meter based on an integrated chip potentiostat (TI LMP91000) was developed to measure the blood glucose levels using a commercial testing strip. As shown in FIG. 10, cyclic voltammetry (CV) was first applied to the commercial testing strip with the bias voltage scanning from −800 mV to 800 mV. The glucose oxidation peak was observed at 150 mV.

FIG. 11A shows a characterization curve of the blood glucose meter based on an integrated chip potentiostat in the glucose detection range of 100 to 550 mg/dL of glucose. Amperometric detection was used to measure the glucose level using an applied bias voltage of 150 mV. The voltage measured at the working electrode by the potentiostat (the x-axis of FIG. 11A) is proportional to the glucose concentration. The output current flow into the working electrode was amplified by a transimpedance and a non-inverting operational amplifier. The amplified signal was fed into an analog-to-digital converter in the all-in-one pen. FIG. 11A shows a positive, linear correlation between the glucose concentration and the voltage.

FIG. 11B shows validation of the developed glucose meter and the comparison with the commercial glucose meter. The developed integrated chip glucose meter accurately measured glucose concentrations of solutions with known glucose concentrations and was virtually indistinguishable from a commercial glucometer. Testing of the glucose meter using commercial test strips indicated that it had comparable performance to a commercial glucometer, as shown in FIG. 11B. A commercially available glucose meter (One Touch Ultra 2) was used to verify and calibrate the blood glucose reading.

### Example Insulin Delivery

After it obtains the blood glucose reading, the all-in-one pen calculates or receives instructions for the size of the insulin dose and then delivers the bolus insulin dose. A bolus calculator may be encoded in either or both of the all-in-one insulin pen system and the external device. The calculation encoded in the all-in-one pen system is responsible for high blood sugar correction based on the blood glucose measurement. The calculation from the external device may consider additional factors from the user, such as carbohydrate intake. The user may manually input an insulin sensitivity factor and an insulin to carbohydrate ratio in the all-in-one pen system and/or the external device as an initialization setting.

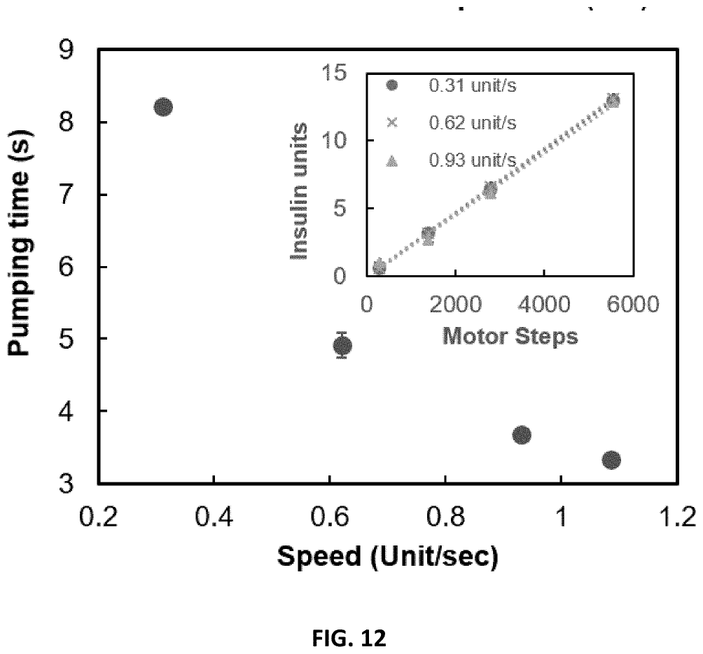

FIG. 12 is a graph of the time used to pump 3 units of insulin in three different swine thighs at various pumping speeds. To confirm accurate dose delivery, the insulin pumps were tested by varying the number of insulin units delivered and the delivery speed via in vitro injection into air. The inset figure in FIG. 12 shows that delivered insulin units were linearly proportional to the controlled motor steps and there was no difference in the accuracy (±2%) of insulin delivery between various speeds. The pumping time dramatically dropped as the pumping speed increased.

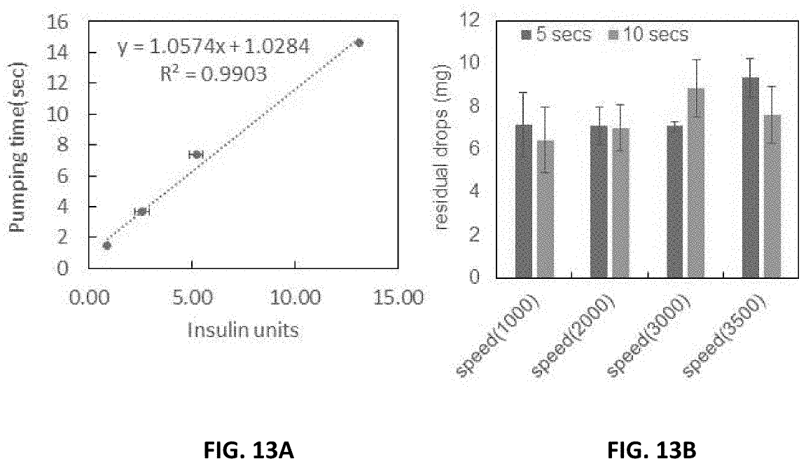

FIG. 13A shows residual drops versus the pumping speed, measured at 5 and 10 seconds after the insulin pump stepper motor in the all-in-one system stops. Standard guidelines for usage of a commercial insulin pen require holding the pen for more than 10 seconds after fully dispensing the insulin to ensure residual drops in the pen are delivered. Similar to commercial insulin pens, residual insulin drops from the all-in-one pen were observed after insulin dose delivery with volumes of less than one unit of insulin. The number of residual drops was independent of pump speed (FIG. 13). Additionally, there was no increase in residual drop size between 5 and 10 seconds after stopping the insulin pump, suggesting that this “hold time” could be decreased, to improve ease of use of the all-in-one system compared to the current standard of care.

FIG. 13B shows pumping time of the developed insulin pump versus the insulin dose. FIG. 13B shows that the pumping time was linearly proportional to the number of units of the insulin dose. The proposed all-in-one vacuum/strip pen system facilitates fully automated procedures of pre-prandial insulin delivery by means of commercially available components, mitigating potential risks and enhancing the success of clinical translation. The automated insulin injection was also performed on pig ears using a 34-gauge needle.

### CONCLUSION

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain, using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of” “only one of” or “exactly one of.” “Consisting essentially of” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An all-in-one insulin pen comprising:
a handheld housing;
a glucose testing module, disposed in the handheld housing, to test a one-time glucose concentration of a patient, the glucose testing module comprising:
a vacuum chamber;
a lancet apparatus; and
a glucose sensor;
an insulin delivery module, disposed in the handheld housing, to automatically administer an insulin bolus to the patient, the insulin delivery module comprising:
an insulin pump; and
an injection motor;
a wireless communication module, disposed in the handheld housing, to communicate with an external device, the external device being capable of (i) setting an insulin dose to be administered by the insulin delivery module and (ii) recording glucose concentration and insulin dose history; and
a microcontroller, disposed in the handheld housing, to initiate and control glucose testing, insulin delivery, and wireless communication.

2. The all-in-one insulin pen of claim 1, wherein the handheld housing has a volume of less than about 300 cubic centimeters.

3. The all-in-one insulin pen of claim 1, wherein the all-in-one insulin pen is configured to calculate an insulin dose using the one-time glucose concentration of the patient.

4. The all-in-one insulin pen of claim 3, wherein:
the wireless communication module is configured to send the insulin dose calculated to the external device; and
the external device is configured to set the insulin dose to be administered using the insulin dose calculated and at least one additional factor input from the patient.

5. The all-in-one insulin pen of claim 1, further comprising a micropump operably coupled to the vacuum chamber to provide a vacuum to the vacuum chamber when the vacuum chamber forms a seal with a portion of skin of the patient.

6. The all-in-one insulin pen of claim 5, wherein the vacuum in the vacuum chamber causes the portion of skin to increase in height by about 2 mm to about 10 mm.

7. The all-in-one insulin pen of claim 6, wherein the vacuum in the vacuum chamber causes the portion of skin to increase in height by about 10 mm.

8. The all-in-one insulin pen of claim 5, wherein the glucose testing module is configured to hold the vacuum for about 3 seconds to about 5 seconds before lancing the portion of skin and about 3 seconds to about 5 seconds after lancing the portion of skin.

9. The all-in-one insulin pen of claim 8, wherein the glucose testing module is further configured to release the vacuum after about 3 seconds to about 5 seconds after lancing, wait 3 seconds to about 5 seconds at atmospheric pressure, and then create the vacuum in the vacuum chamber again.

10. The all-in-one insulin pen of claim 1, wherein the lancet apparatus comprises a spring-loaded actuator.

11. The all-in-one insulin pen of claim 1, wherein the lancet apparatus comprises a motorized lancet.

12. The all-in-one insulin pen of claim 11, wherein the lancet apparatus comprises at least one of a solenoid actuator or a DC-micro motor.

13. The all-in-one insulin pen of claim 1, wherein the external device initializes the all-in-one insulin pen.

14. The all-in-one insulin pen of claim 1, wherein:
the glucose testing module further comprises a single-use testing strip; and
the insulin delivery module further comprises a single-use needle.

15. The all-in-one insulin pen of claim 1, wherein the glucose testing module is configured to extract at least 1 microliter of blood from the patient.

16. A method of making an all-in-one insulin pen, the all-in-one insulin pen comprising:
a vacuum chamber;
a micropump;
a lancet apparatus;
a glucose sensor;
an insulin pump;
an injection motor;
a wireless communication module;
a microcontroller unit;
a handheld housing; and
the method comprising:
operably coupling the microcontroller unit with the micropump, lancet apparatus, glucose sensor, insulin pump, injection motor, and wireless communication module;
operably coupling the micropump with the vacuum chamber;
operably coupling the insulin pump with the injection motor; and
assembling the all-in-one insulin pen into the handheld housing.

17. A method of using an all-in-one insulin pen, the all-in-one insulin pen comprising:
a handheld housing;
a glucose testing module disposed within the handheld housing;
an insulin delivery module disposed within the handheld housing; and
the method comprising:
placing the all-in-one insulin pen against a portion of skin of a user; and
actuating the all-in-one insulin pen;
wherein, while the all-in-one insulin pen is continuously held against the portion of skin:
a one-time blood glucose concentration of the user is tested by the glucose testing module;
an insulin dose is calculated based on the blood glucose concentration; and
the insulin dose is administered to the user by the insulin delivery module.

18. The method of claim 17, wherein the glucose testing module further comprises:
a vacuum chamber to form a seal against the portion of skin; and
a micropump to form a vacuum in the vacuum chamber while the one-time blood glucose concentration of the user is tested.

19. The method of claim 18, wherein the vacuum causes the portion of skin to increase in height by about 2 mm to about 10 mm.
